(19) United States
(12) Patent Application Publication
(10) Pub. No.: US 2023/0255307 A1
(43) Pub. Date: Aug. 17, 2023

(54) FOOTWEAR AND INSERT HAVING THERAPEUTIC LIGHT SOURCE

(71) Applicant: Biothread LLC, Wayne, PA (US)

(72) Inventors: Jay Tapper, Wayne, PA (US); Daniel Bish, New York, NY (US); Daniel Shuter, New York, NY (US); Kristien Del Ferraro, Stamford, CT (US)

(21) Appl. No.: 17/671,764

(22) Filed: Feb. 15, 2022

(51) Int. Cl. A43B 3/36; A43B 1/10; A43B 7/00

(52) U.S. Cl. CPC A43B 3/36; A43B 1/10; A43B 7/00

(57) ABSTRACT

An insole for an article of footwear includes an insole support and a side-emitting optical light tube on the insole support member. The insole support member includes a top surface and a bottom surface where the top surface faces towards a wearer’s foot when the insole is positioned in the article of footwear and the article of footwear is worn. The side-emitting optical light tube is configured to project radiation having a therapeutic wavelength through the side-emitting optical tube and toward the wearer’s foot. The side-emitting optical light tube includes a side-emitting optical fiber and a jacket made from a light-transmissive material at least partially surrounding the side-emitting optical fiber.

### BACKGROUND

Articles of footwear often include both an upper and a sole. The upper encloses a wearer’s foot, and in some circumstances provides support for the foot during motion. The sole generally provides traction, protection, and also can support the foot. Typically, an article of footwear also includes an insole placed within the upper between the wearer’s foot and the sole to provide additional comfort as well as increased performance for various activities.

Articles of footwear typically do not include lights. For footwear having lights prior art patent documents are typically directed to footwear where the light is directed outward to produce a warning effect or enhance the outward visual effect of the shoe. US 10,806,211 B1 discloses an article of footwear including a light delivery system that is configured to project light toward the foot, ankle and/or leg of the wearer; however, further improvements can be made.

### SUMMARY

In view of the foregoing, an insole for an article of footwear includes an insole support and a side-emitting optical light tube on the insole support member. The insole support member includes a top surface and a bottom surface where the top surface faces towards a wearer’s foot when the insole is positioned in the article of footwear and the article of footwear is worn. The side-emitting optical light tube is configured to project radiation having a therapeutic wavelength through the side-emitting optical tube and toward the wearer’s foot. The side-emitting optical light tube includes a side-emitting optical fiber and a jacket made from a light-transmissive material at least partially surrounding the side-emitting optical fiber.

An article of footwear can include an upper, a sole connected to the upper, and the insole described above.

Additionally, an insole for an article of footwear can include an insole support member, a side-emitting optical fiber and an insole liner. The insole support member has a top surface and a bottom surface where the top surface faces towards a wearer’s foot when the insole is positioned in the article of footwear and the article of footwear is worn. The insole support member further defines a channel. The side-emitting optical fiber is received in the channel. The insole liner, which is light-transmissive material in at least one area above the fiber, is positioned at least partially over the side-emitting optical fiber and the top surface.

Moreover, an article of footwear can include an upper, a sole connected to the upper, and the insole described above.

### BRIEF DESCRIPTION OF THE DRAWINGS

FIG. 1 is an upper perspective view of an insole assembly.

FIG. 2 is an upper perspective view of an insole support member.

FIG. 3 is a lower perspective view of the insole support member.

FIG. 4 is an upper perspective view of the insole support member and a side-emitting optical light tube on the insole support member.

FIG. 5 is a cross-sectional view taken through line 5-5 of FIG. 4.

FIG. 6 is an alternative side-emitting optical light tube shown in cross section.

FIG. 7 is an upper perspective view of the insole support member and the side-emitting optical light tube on the insole support member prior to placement of an insole liner on the insole support member.

FIG. 8 is a schematic depiction of a light pod for the insole assembly of FIG. 1.

FIG. 9 is a side view of an article of footwear and the light pod.

FIG. 10 is an upper perspective view of an insole assembly including an extension.

FIG. 11 is an upper view of an article of footwear with the insole assembly of FIG. 10.

### DETAILED DESCRIPTION

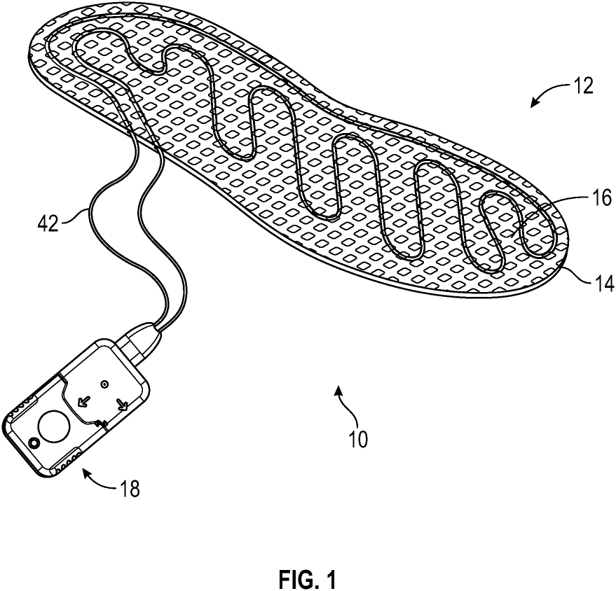

Referring to FIG. 1, an insole assembly 10 according to at least one embodiment described herein may be adapted for placement in any kind of footwear, including, but not limited to: running shoes, hiking boots, soccer shoes, football shoes, sneakers, rugby shoes, basketball shoes, baseball shoes as well as other kinds of non-athletic shoes, including, but not limited to: dress shoes, loafers, sandals, and boots. The insole assembly 10 may include provisions for improving support for a foot placed within an article of footwear. The insole assembly 10 also includes provisions for operating as a light (or radiation) delivery system in that it is configured to project light (or radiation) toward the foot of the wearer. Light having a wavelength between 630 nm and 900 nm has been found beneficial to increase blood flow, may provide ameliorative effects with regard to inflammation, and can be beneficial in the treatment of diabetic neuropathy, and as such can be referred to as light having a therapeutic wavelength. The insole assembly 10 may be configured, however, to project light at wave lengths other than between 630 nm and 900 nm, which also may have a therapeutic effect.

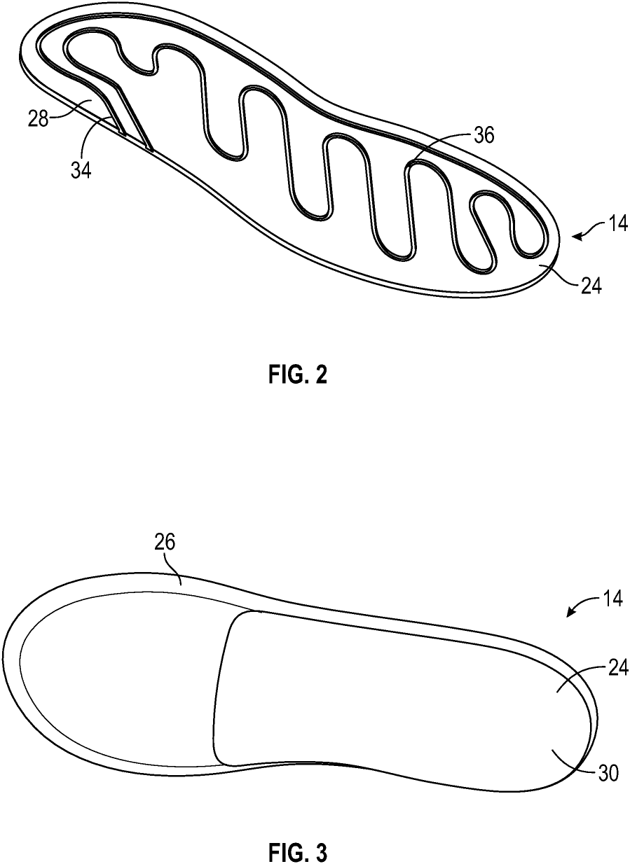

The insole assembly 10 includes an insole 12 including an insole support member 14, which may be made of a cushioning material, and an insole liner 16. The insole assembly 10 also includes a light pod 18 to provide the light source capable of providing the therapeutic wavelength. In some embodiments, the insole support member 14 exhibits a layered or partially layered structure. For example and with reference to FIGS. 2 and 3, the insole support member 14 may include a first layer 24 of material and a second layer 26 of material as well as other layers or inserts (not shown), if desired. The insole support member 14 may also include only the first layer 24. The insole support member 14 may have the overall shape and dimensions of a traditional footbed to allow the insole 12 to be placed into the footbed of an article of footwear.

The first layer 24 and/or a second layer 26 or other layers or inserts (if provided) can be formed from a cushioning material, such as an EVA resin foam or a soft polyethylene foam. Different layers may be made from different materials, e.g., the first layer 24 may be made from a first material having a first hardness level and the second layer 26 may be made from a second material having a second hardness level.

With reference to FIGS. 2 and 3, the insole support member 14, whether being one layer, multiple layers or including inserts, has a top surface 28 and a bottom surface 30 (see FIG. 3). The top surface 28 faces towards a wearer’s foot when the insole 12 is positioned in an article of footwear and the article of footwear is worn. The insole support member 14 can at least partially define a channel 34 (multiple similarly shaped channels could also be provided) extending downwardly from the top surface 28 toward the bottom surface 30. In other embodiments, however, the channel may not be provided in the insole support member 14. When provided, the channel 34 can define a channel floor 36. When provided, the channel floor 36 is offset downwardly from the top surface 28. In some circumstances the channel floor 36 can be offset upwardly from the bottom surface 30. In other circumstances, the channel 34 can extend through the first layer 24 into the second layer 26 or another layer or insert provided with the insole support member 14 so that the channel floor 36 is provided in the second layer 26 or another layer or insert. In yet some other circumstances, the channel 34 may extend entirely through the insole support member 14.

When provided, the channel floor 36 can be offset downwardly from the top surface 28 to provide the channel 34 with a depth d (see FIG. 5) between 0.25 mm and 4 mm. When the channel 34 extends entirely through the insole support member 14, the channel 34 can have a depth d measured downwardly from the top surface 28 even greater than 4 mm. The channel 34 can have a width w (see FIG. 5) measured perpendicular to the depth d between 0.25 mm and 4 mm. In FIG. 5 the channel floor 36 is depicted as residing in a plane, however, the channel floor 36 could be curved.

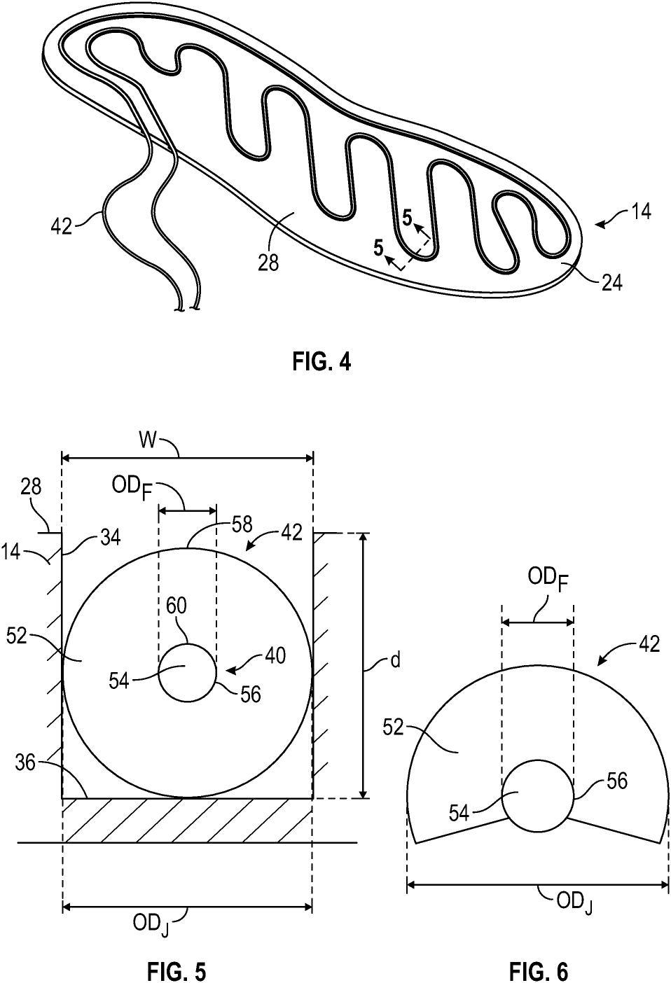

With reference to FIGS. 4 - 6, the insole assembly 10 further includes a side-emitting optical fiber 40, which can be provided as part of a side-emitting light tube 42, or can be provided by itself, e.g., not part of the side-emitting light tube 42. If desired, the insole assembly 10 may include multiple side-emitting optical fibers 40 and/or multiple side-emitting light tubes 42. When provided, the side-emitting light tube 42 includes the side-emitting optical fiber 40 or multiple side-emitting optical fibers 40 and a jacket 52 at least partially surrounding the side-emitting optical fiber 40 or multiple side-emitting optical fibers 40. The side-emitting optical fiber 40 includes a core 54 and cladding 56 surrounding the core 54. The core 54 in the illustrated embodiment is made from a synthetic polymeric material, e.g., a high-purity polymethyl methacrylate (PMMA). The cladding 56 in the illustrated embodiment is made from a fluorinated polymer. The jacket 52 in the illustrated embodiment is made from silicone, which can be light-transmissive, having a durometer of at least 50A, and preferably at least 65A. The jacket 52 can be extruded over the side-emitting optical fiber 40 such that the jacket 52 surrounds the side-emitting optical fiber 40 and is in contact with the cladding 56. Even though the jacket 52 is in contact with the cladding 56 so that no or a very small air gap is provided between them, the side-emitting optical fiber 40 may be movable with respect to the jacket 52 in an axial direction, which is parallel with a longest dimension of the side-emitting light tube 42. Allowing for movement of the side-emitting optical fiber 40 with respect to the jacket 52 can be helpful in preventing damage to the side-emitting optical fiber 40 when the side-emitting light tube 42 is bent.

The maximum outer dimension of the jacket 52, which will be referred to as an outer diameter (OD_{J}) although the jacket 52 need not be circular in cross-section but instead could be U-shaped (see FIG. 6), for example, is at least four times, and preferably at least six times, the outer diameter (OD_{F}) of the side-emitting optical fiber 40. As an example, the outer diameter (OD_{F}) of the side-emitting optical fiber 40 can be 0.25 mm and the outer diameter (OD_{J}) of the jacket 52 between 1.0 mm and 1.5 mm. The side-emitting optical fiber 40 could have a smaller outer diameter (OD_{F}). As another example, the outer diameter (OD_{F}) of the side-emitting optical fiber 40 can be 0.5 mm with the outer diameter (OD_{J}) of the jacket 52 being between two times to three times greater than the maximum outer dimension (OD_{F}) of the side-emitting optical fiber 40. Having the outer diameter (OD_{F}) of the side-emitting optical fiber 40 larger than 0.5 mm with the relatively larger jacket 52 can result in the side-emitting light tube 42 being too large in cross-section making it difficult to incorporate into the insole assembly 10 in an aesthetic and practically functional manner. Providing the jacket 52 with the maximum outer dimension (OD_{J}) that is between two times to six times greater that the outer diameter (OD_{F}) of the side-emitting optical fiber 40 protects the side-emitting optical fiber 40 against excessive force and dropping.

During assembly, the side-emitting light tube 42 (or only the side-emitting optical fiber 40) can be pressed into the channel 34 so that the side-emitting light tube 42 (or only the side-emitting optical fiber 40) is received in the channel 34. With particular reference to FIG. 5, an uppermost surface 58 on the side-emitting light tube 42 and/or an uppermost surface 60 on the side-emitting optical fiber 40 may be offset beneath the top surface 28. As illustrated in FIG. 5, the uppermost surface 58 on the side-emitting light tube 42 is offset beneath the top surface 28. The maximum outer dimension (OD_{J}) of the jacket 52 may be less than the depth (d) of the channel 34. The maximum outer dimension (OD_{J}) of the jacket 52 may also be less than or equal to the width of the channel 34. Where the maximum outer dimension (OD_{J}) of the jacket 52 is equal to the width of the channel 34, the side-emitting light tube 42 may be retained within the channel 34 via frictional engagement with the insole 12, and more particularly, via frictional engagement with the insole support member 14. Likewise, where only the side-emitting optical fiber 40 is provided, e.g., no jacket 52 is provided around the side-emitting optical fiber 40, the side-emitting optical fiber 40 may have an outer diameter (OD_{F}) less than the depth (d) of the channel 34 and less than or equal to the width of the channel 34. The insole liner 16 may also retain the side-emitting optical fiber 40 and/or the side-emitting light tube 42 within the channel 34, and an adhesive may also be used to retain the side-em itting optical fiber 40 and/or the side-emitting light tube 42 within the channel 34.

When no channel is provided in the insole support member 14, the side-emitting optical fiber 40 and/or the side-emitting light tube 42 may be retained to the top surface 28 of the insole support member 14 via adhesive and/or stitches, e.g. embroidery stitches. Additionally, the insole liner 16 may also retain the side-emitting optical fiber 40 and/or the side-emitting light tube 42 to the top surface 28 of the insole support member 14 when no channel is provided in the insole support member 14.

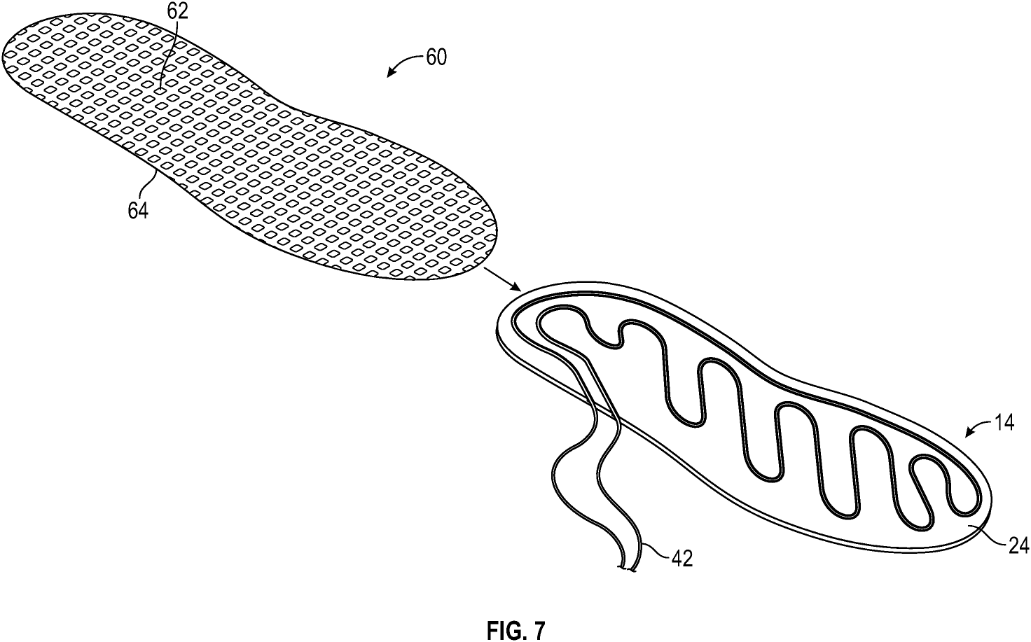

With reference to FIGS. 1 and 7, the insole assembly 10 further includes the insole liner 16, which is light-transmissive. The insole liner 16 may include additional properties that may be desirable. For example, the insole liner 16 may comprise a cloth or fabric material in some embodiments. In other embodiments, the insole liner 16 may comprise a mesh (fabric or plastic) material and/or a plastic film. With reference to FIG. 7, the insole liner 16 includes an upper side 62 and a lower side 64. The lower side 64 of the insole liner 16 is disposed on the top surface 28 of the first layer 24. The upper side 62 of insole liner 16 is disposed closest to a wearer’s foot when the insole 12 is located within an article of footwear. The insole liner 16 may adhered to the top surface 28 of the insole support member 14. In the illustrated embodiment, the periphery of the insole liner 16 matches the periphery of the insole support member 14. This can allow the insole 12 to have an overall shape with a periphery conforming to a traditional footbed of an article of footwear. If desired, however, the insole liner 16 need not match the periphery of the insole support member 14, but instead may only cover the side-emitting light tube 42 or the side-emitting optical fiber 40 on the insole support member 14. As such, the insole liner 16 can be light-transmissive in at least one area above the side-emitting light tube 42 or the side-emitting optical fiber 40 and positioned at least partially over the side-emitting light tube 42 or the side-emitting optical fiber 40 and the top surface 28. In other words, the entirety of the top surface 28 and the entirety of the side-emitting light tube 42 or the side-emitting optical fiber 40 need not be covered by the insole liner 16.

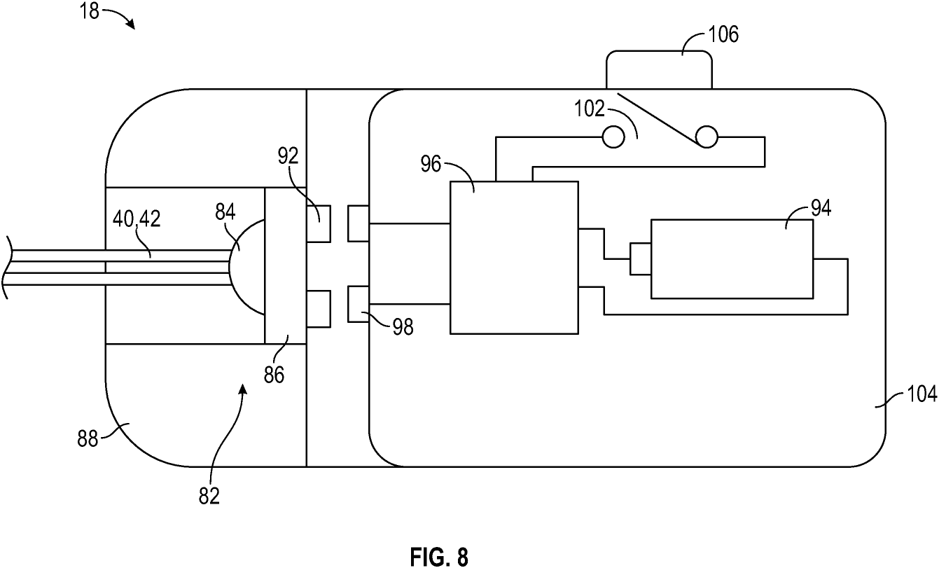

With reference back to FIG. 1, the insole assembly 10 further includes a light pod 18. FIG. 8 is a schematic depiction of the light pod 18 in which an optical fiber light source 82, which can be a laser diode 84 mounted to a circuit board 86, are both received in the light source housing 88. Another type of light source, e.g., an LED, could be used instead of the laser diode. Also, the circuit board 86 need not be a rigid board, but could also be a flexible support with appropriate circuitry. Light source electrical contacts 92 extend from the circuit board 86 for receiving power. A power source, such as a battery 94 is in electrical communication with a controller 96, which is electrically connected with power source electrical contacts 98. The controller 96 can be an appropriate integrated circuit, for example. The controller 96 is in electrical communication with a switch 102 that can control power delivery to the controller 96 and the optical fiber light source 82. The battery 94, the controller 96 and the switch 102 can be received in a power source housing 104, and an actuator 106, e.g., a button, can be accessible to a user on the outside of the power source housing 104 for operating the switch 102. The light source electrical contacts 92 can connect with the power source electrical contacts 98 when the power source housing 104 is connected with the light source housing 88. The optical fiber light source 82 optically couples to the side-emitting light tube 42 or the side-emitting optical fiber 40 so as to project light into the side-emitting optical fiber 40.

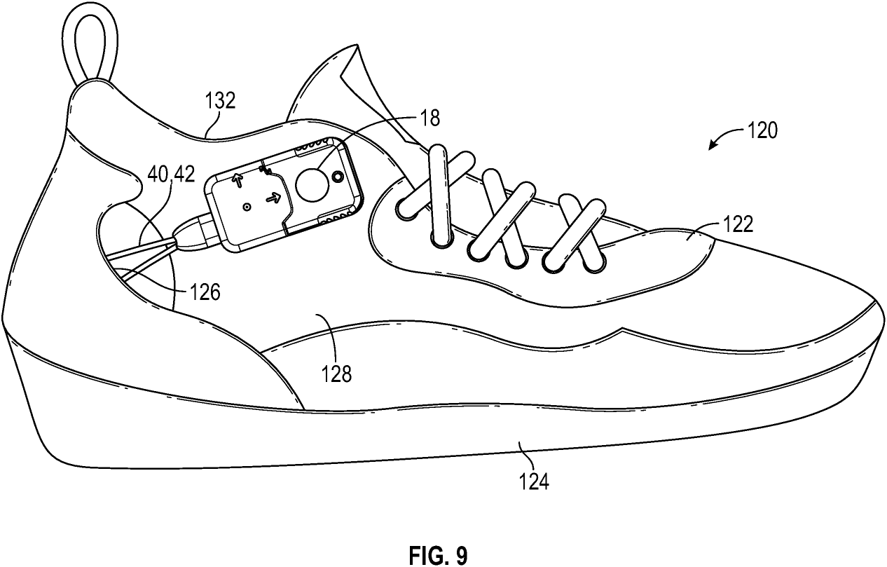

FIG. 9 depicts an article of footwear 120 having an upper 122 and a sole 124. The upper 122 is generally designed to enclose a wearer’s foot, and in some circumstances to provide support for the foot during motion. The sole 124 is generally designed to provide traction, protection, and also to support the foot. The insole 12 (not visible in FIG. 9) is placed within the upper 122 between the wearer’s foot and the sole 124 in a similar manner to a conventional insole. The upper 122 also includes a passage 126, which can be a slot or a hole extending through the upper 122 from inside the upper 122 to an outer surface 128 of the article of footwear 120. The passage 126 in the embodiment illustrated in FIG. 9 is distinct and offset from an opening 132 in the article of footwear 120 that receives the wearer’s foot when the article of footwear 120 is put on. The light pod 18 is configured to attach to the outer surface 128 of the article of footwear 120, and more particularly in the illustrated embodiment to the outer surface 128 of the upper 122. The side-emitting light tube 42 and/or the side-emitting optical fiber 40 extends through the passage 126.

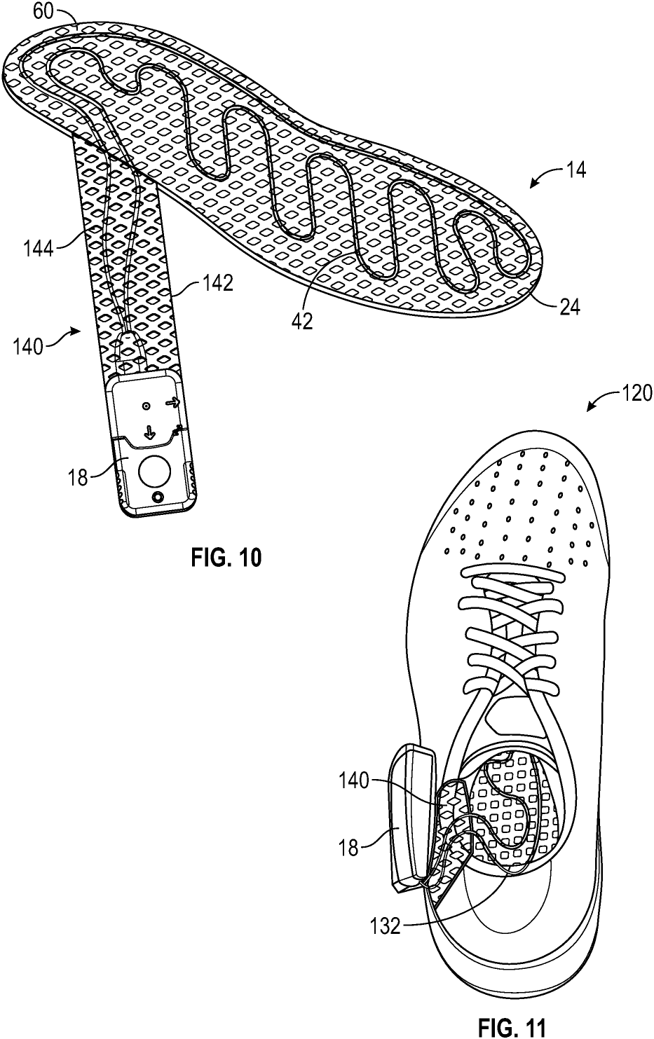

FIG. 10 depicts the insole assembly 10 further including an extension 140. The extension 140 extends from the periphery of the insole support member 14. With reference to FIG. 11, the extension 140 is configured to extend outwardly from the opening 132 in the article of footwear 120 configured to receive a wearer’s foot. The extension 140 can be made from a strip 142 of material that is a material similar to the material from which the insole support member 14 is made. An extension liner 144, which can be made from a material similar to the material from which the insole liner 16 is made, can cover an upper surface of the strip 142. The side-emitting light tube 42 or the side-emitting optical fiber 40 can be fixed to the extension 140, e.g. the strip 142 may or may not have channels similar to the channel 34, and the side-emitting light tube 42 or the side-emitting optical fiber 40 can be received in the channels. Also, the extension liner 144 can cover the side-emitting light tube 42 or the side-emitting optical fiber 40. The light pod 18 may be fixed to the extension 140.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. An insole for an article of footwear comprising:
an insole support member having a top surface and a bottom surface, the top surface facing towards a wearer’s foot when the insole is positioned in the article of footwear and the article of footwear is worn; and
a side-emitting optical light tube on the insole support member, the side-emitting optical light tube configured to project radiation having a therapeutic wavelength through the side-emitting optical light tube and toward the wearer’s foot, the side-emitting optical light tube including a side-emitting optical fiber and a jacket, which is light-transmissive, at least partially surrounding the side-emitting optical fiber.

2. The insole of claim 1, wherein the insole support member includes a channel, and the side-emitting optical light tube is received in the channel.

3. The insole of claim 2, wherein the channel defines a channel floor offset downwardly from the top surface and a lowermost portion of the channel floor is offset downwardly from the top surface between 0.25 mm to 4 mm.

4. The insole of claim 3, wherein an uppermost surface on the side-emitting optical fiber is offset beneath the top surface.

5. The insole of claim 2, wherein the insole support member includes a first layer comprised of a first material having a first hardness level and an additional layer or insert comprised of a second material having a second hardness level, and the channel extends into the additional layer or insert.

6. The insole of claim 1, wherein the jacket has a maximum outer dimension between 0.5 mm and 3 mm.

7. The insole of claim 6, wherein the outer diameter of the side-emitting optical fiber is at least 0.25 mm and less than or equal to 0.5 mm.

8. The insole of claim 1, wherein the jacket is circular in cross section.

9. The insole of claim 1, wherein the light-transmissive material from which the jacket is made is silicone.

10. The insole of claim 9, wherein the light-transmissive material from which the jacket is made has a durometer of at least 50A.

11. The insole of claim 1, wherein the side-emitting optical fiber is movable with respect to the jacket in an axial direction, which is parallel with a longest dimension of the side-emitting optical light tube.

12. The insole of claim 1, wherein the insole support member has an overall shape with a periphery conforming to a traditional footbed of an article of footwear.

13. The insole of claim 12, further comprising an insole liner, which is made from a light-transmissive material, positioned at least partially over the top surface, wherein the insole liner has an overall shape and periphery matching an overall shape and periphery of the top surface of the insole support member.

14. The insole of claim 1, further comprising an extension extending from a periphery of the insole support member, the extension configured to extend outwardly from an opening in the article of footwear configured to receive a wearer’s foot.

15. The insole of claim 14, wherein the side-emitting optical light tube is fixed to the extension.

16. The insole of claim 1, further comprising an insole liner, which is made from a light-transmissive material, positioned at least partially over the top surface.

17. The insole of claim 16, wherein the insole liner has an overall shape and periphery matching an overall shape and periphery of the top surface of the insole support member.

18. The insole of claim 16, wherein the insole liner is a plastic film, a mesh or a fabric.

19. The insole of claim 1, in combination with a light pod including a light source, wherein the side-emitting optical tube is optically connected or optically connectable with the light source.

20. The insole of claim 19, in combination with an article of footwear, wherein the light pod is configured to attach to an outer surface of the article of footwear.

21. An article of footwear comprising:
an upper;
a sole connected to the upper; and
the insole of claim 1.

22. The article of footwear of claim 21, further comprising a light pod including a light source, wherein the side-emitting optical light tube is optically connected to or optically connectable with the light source.

23. The article of footwear of claim 21, wherein the upper includes an opening for receiving a wearer’s foot and a passage extending from inside the upper to an exterior through which the side-emitting optical light tube passes.

24. The article of footwear of claim 23, further comprising a light pod including a light source, wherein the side-emitting optical tube is optically connected to or optically connectable with the light source wherein the light pod is configured to attach to an outer surface of the upper.

25. An insole for an article of footwear comprising:
an insole support member having a top surface and a bottom surface, the top surface facing towards a wearer’s foot when the insole is positioned in the article of footwear and the article of footwear is worn, the insole support member further defining a channel;
a side-emitting optical fiber received in the channel; and
an insole liner, which is light-transmissive in at least one area above the side-emitting optical fiber, positioned at least partially over the side-emitting optical fiber and the top surface.

26. The insole of claim 25, wherein the channel defines a channel floor offset downwardly from the top surface and a lowermost portion of the channel floor is offset downwardly from the top surface between 0.25 mm to 4 mm.

27. The insole of claim 25, wherein the insole support member includes a first layer comprised of a first material having a first hardness level and an additional layer or insert comprised of a second material having a second hardness level, and the channel extends into the additional layer or insert.

28. The insole of claim 25, wherein the side-emitting optical fiber is a side-emitting optical light tube including the side-emitting optical fiber and a jacket made from a light-transmissive material at least partially surrounding the side-emitting optical fiber.

29. The insole of claim 28, wherein the jacket has a maximum outer dimension between 0.5 mm and 3 mm.

30. The insole of claim 29, wherein the outer diameter of the side-emitting optical fiber is at least 0.25 mm and less than or equal to 0.5 mm.

31. The insole of claim 30, wherein the jacket is circular in cross section.

32. The insole of claim 31, wherein the light-transmissive material from which the jacket is made is silicone.

33. The insole of claim 32, wherein the light-transmissive material from which the jacket is made has a durometer of at least 50A.

34. The insole of claim 33, wherein the side-emitting optical fiber is movable with respect to the jacket in an axial direction, which is parallel with a longest dimension of the side-emitting optical light tube.

35. The insole of claim 25, wherein the insole support member has an overall shape with a periphery conforming to a traditional footbed of an article of footwear.

36. The insole of claim 35, wherein the insole liner has an overall shape and periphery matching the overall shape and the periphery of the top surface of the insole support member.

37. The insole of claim 25, further comprising an extension extending from a periphery of the insole support member, the extension configured to extend outwardly from an opening in the article of footwear configured to receive a wearer’s foot.

38. The insole of claim 37, wherein the side-emitting optical fiber is fixed to the extension.

39. The insole of claim 25, in combination with a light pod including a light source, wherein the side-emitting optical fiber is optically connected or optically connectable with the light source.

40. The insole of claim 39, in combination with an article of footwear, wherein the light pod is configured to attach to an outer surface of the article of footwear.

41. An article of footwear comprising:
an upper;
a sole connected to the upper; and
the insole of claim 25.

42. The article of footwear of claim 41, further comprising a light pod including a light source, wherein the side-emitting optical fiber is optically connected to or optically connectable with the light source.

43. The article of footwear of claim 41, wherein the upper includes an opening for receiving a wearer’s foot and a passage extending from inside the upper to an exterior through which the side-emitting optical fiber passes.

44. The article of footwear of claim 43, further comprising a light pod including a light source, wherein the side-emitting optical fiber is optically connected to or optically connectable with the light source wherein the light pod is configured to attach to an outer surface of the upper.
